SEQUENCE LISTING (3 sequences)

SEQ ID NO: 1 - protein, 26 aa, synthetic construct
GRKKRRQRRRPQQAQFGRGGWQPFHK

SEQ ID NO: 2 - protein, 27 aa, synthetic construct
GRKKRRQRRRPQQAQFGRGRGWQPFHK

SEQ ID NO: 3 - protein, 12 aa, synthetic construct
GRKKRRQRRRPQ

(19) United States
(12) Patent Application Publication
(10) Pub. No.: US 2025/0000952 A1
(43) Pub. Date: Jan. 2, 2025

(54) POLYPEPTIDE BASED ON RECEPTOR-INTERACTING PROTEIN KINASE-3 METHYLATION MODIFICATION AND APPLICATION THEREOF IN CELL NECROPTOSIS-RELATED DISEASES

(71) Applicant: Air Force Medical University of the People's Liberation Army, Xi'an City (CN)

(72) Inventors: Jian Zhang, Xi'an City (CN); Pan Zhao, Xi'an City (CN); Suning Chen, Xi'an City (CN)

(73) Assignee: Air Force Medical University of the People's Liberation Army, Xi'an City (CN)

(21) Appl. No.: 18/614,084

(22) Filed: Mar. 22, 2024

(30) Foreign Application Priority Data
Jun. 30, 2023 (CN) 202310795490.8

(51) Int. Cl. A61K 38/45; A61K 38/16; A61P 1/00; C07K 14/005; C12N 9/12

(52) U.S. Cl. CPC A61K 38/45; A61K 38/162; A61P 1/00; C07K 14/005; C12N 9/12; C12Y 207/11001

(57) ABSTRACT

A polypeptide based on RIP3 methylation modification and application thereof in cell necroptosis-related diseases are provided, belonging to the technical field of biomedicine. The polypeptide for inhibiting intestinal inflammatory injury (TAT-mRIP3-SDMA) is formed by connecting cell-penetrating peptide TAT and methylated RIP3 polypeptide.

### CROSS-REFERENCE TO RELATED APPLICATIONS

This application claims priority of Chinese Patent Application No. 202310795490.8. filed on Jun. 30, 2023, the entire contents of which are incorporated herein by reference.

### INCORPORATION BY REFERENCE OF SEQUENCE LISTING

The Sequence Listing XML associated with this application is provided electronically in XML file format and is hereby incorporated by reference into the specification. The name of the XML file containing the Sequence Listing is “2024-0258.xml”. The XML file is 3,664 bytes, created on Mar. 14, 2024, and is being submitted electronically via USPTO Patent Center.

### TECHNICAL FIELD

The disclosure belongs to the technical field of biomedicine, and particularly relates to a polypeptide based on Receptor-Interacting Protein Kinase-3 (RIP3) methylation modification and application thereof in cell necroptosis-related diseases.

### BACKGROUND

Mammalian intestine is a highly complex ecosystem, which plays key functions such as digestion, absorption and assimilation, as well as accommodating and hosting a large number of symbiotic microorganisms. Maintaining this extensive functional system requires a complete and precise regulation mechanism for intestinal homeostasis. As the first defense barrier of the intestine, intestinal epithelial cells undergo renewal every 4-7 days on average to maintain the balance of the intestinal homeostasis. Therefore, the renewal and death of intestinal epithelial cells is extremely important to maintain the integrity of defense barrier and the intestinal homeostasis Studies on animal models and clinical patients have confirmed that abnormal death of intestinal epithelial cells leads to inflammatory bowel disease (IBD) and preclinical colitis. Therefore, it is very important to study the death mechanism of intestinal epithelial cells for the maintenance of intestinal homeostasis and the occurrence and development of diseases.

Under normal homeostasis conditions, intestinal epithelial cells activate the mechanism of regeneration and repair after being damaged. The phagocytosis of dead cells, apoptotic bodies produced by dead cells or secreted regulatory factors activate the regeneration of intestinal epithelial cells, thus restoring the balance of intestinal homeostasis. Therefore, the precise regulation of the death mode of intestinal epithelial cells is the key to maintain intestinal homeostasis. As an important type of intestinal epithelial cell death, necroptosis plays an important role in maintaining intestinal homeostasis. Previous studies have shown that the dysfunction of Caspase-8, RIPK1 (RIP1), RIPK3 (RIP3) and other key molecules of cell necroptosis may induce imbalance of intestinal homeostasis and intestinal inflammation. Protein Arginine Methyltransferases (PRMTs) family members regulate a variety of biological processes by catalyzing the methyl group on adenosylmethionine to connect to the arginine residue of the target protein, so that the substrate protein is methylated. As an important type-II PRMTs member, Protein Arginine Methyltransferase 5 (PRMT5) may catalyze symmetric di-methylation of histone and non-histone arginine residues, and participate in the regulation of various biological processes, such as genome stability, RNA splicing, cell cycle, cell proliferation, apoptosis and differentiation.

Necroptosis, which was formally proposed by scientists in 2005, is different from traditional cell necrosis. Closely related to inflammation, necroptosis is precisely regulated by signal molecular pathway and is widely involved in the development and steady state maintenance of the body. Different from the morphological changes caused by apoptosis, cells undergoing necroptosis exhibit round, the cytoplasm swells, the cell permeability changes, chromatin does not agglutinate, the cell membrane eventually breaks, the contents leak out and subsequent cell death. Many cell death receptors, such as tumor necrosis factor receptor 1 (TNFR1) can cause necroptosis by binding and activating their respective cytoplasmic connexin. Necroptosis is a precisely regulated death pathway. Tumor cell necrosis factor TNF-α may induce receptor-interacting serine/threonine protein kinases 1 and 3 (RIPK1, RIPK3) and effector MLKL, and the phosphorylation level of RIP1, RIP3 and MLKL increases, resulting in cell death. The gene sequences of RIPK1 (RIP1), RIPK3 (RIP3) and MLKL, which are key molecules for regulating necroptosis, are highly homologous in human and mouse. The full length of mouse RIP1 protein has 656 amino acids, which consists of N-terminal kinase domain, middle domain and C-terminal death domain. RIP1 binds to molecules (such as TRADD, FADD, TNFR1) through the C-terminal death domain, and participates in signal transduction after stimulation of TNF, FasL, TRAL, etc., and induces cell apoptosis. Necroptosis is induced by the binding with RIP3 through RHIM domain.

More and more studies show that necroptosis is closely related to the immune system, nervous system and occurrence and development of inflammation. Therefore, understanding the regulation mechanism of signaling pathway for necroptosis and its physiological and pathological significance has profound implications on the prevention and treatment of human related diseases. At present, a number of small molecular inhibitors targeting RIP1 and RIP3, the key molecules of necroptosis, are in the process of research and development, but no mature drugs have been available in the clinic. Polypeptide drugs targeting necroptosis are helpful to provide new research and development strategies. Therefore, polypeptide targeted intervention drugs targeting the signal pathway for necroptosis are expected to be applied to the treatment of cell necroptosis-related diseases such as autoimmune, inflammation and neurodegenerative diseases.

### SUMMARY

The objective of the present disclosure is to provide a polypeptide based on RIP3 methylation modification and application thereof in cell necroptosis-related diseases.

In order to achieve the above objectives, the disclosure adopts the following technical scheme.

The disclosure discloses a polypeptide based on RIP3 methylation modification, and the polypeptide is formed by connecting cell-penetrating peptide TAT and methylated RIP3 polypeptide.

Optionally, a 479th arginine of RIP3 protein is subjected to symmetric di-methylation modification.

Optionally, an amino acid sequence of the polypeptide based on RIP3 methylation modification is shown in SEQ ID NO:1.

Optionally, a molecular weight of the polypeptide is 3821.27 Dalton (Da).

The present disclosure further provides an application of the polypeptide based on RIP3 methylation modification mentioned above in preparing a medicine for preventing and/or treating diseases caused by cell necroptosis.

Optionally, the medicine is a medicine for increasing a resistance of cells to necroptosis.

More optionally, the medicine is a medicine for reducing cell death and necroptosis.

More optionally; the medicine is a medicine for reducing expression levels of necroptosis molecules pRIP1, pRIP3 and pMLKL.

The present disclosure further provides an application of the polypeptide based on RIP3 methylation modification mentioned above in preparing a medicine for preventing and/or treating intestinal inflammatory injury.

The present disclosure further provides a medicine for inhibiting intestinal inflammatory injury, the medicine is prepared by the polypeptide based on RIP3 methylation modification mentioned above with or without pharmaceutically acceptable adjuvants.

Compared with the prior art, the disclosure has the following beneficial effects.

The disclosure discloses a polypeptide TAT-mRIP3-SDMA (the amino acid sequence is shown in SEQ ID NO:1) based on RIP3 methylation modification, the polypeptide TAT-mRIP3-SDMA is formed by connecting the cell-penetrating peptide TAT and the methylated RIP3 polypeptide. Experiments prove that the TAT-mRIP3-SDMA polypeptide is capable of entering mouse lung fibroblast L929 (a classic cell model for cell necroptosis study), increasing the cell's capability to resist necroptosis induced by TNF-α, and reducing cell death: decreasing the expression levels of key molecules of necroptosis such as pRIP1, pRIP3 and pMLKL. The polypeptide is easy to synthesize artificially, and has practical application and popularization value in the protection of intestinal inflammatory injury and the treatment of diseases caused by cell necroptosis.

### BRIEF DESCRIPTION OF THE DRAWINGS

FIG. 1A is an immunofluorescence analysis of fluorescein isothiocyanate (FITC)-labeled polypeptide TAT-mRIP3 entering cell.

FIG. 1B is an immunofluorescence analysis of FITC-labeled polypeptide TAT-mRIP3-SDMA entering cell.

FIG. 2 shows a morphological analysis of cells after intervention of cell necroptosis combined with polypeptide.

FIG. 3 shows an expression detection of a marker molecule in cell necroptosis (cell level).

FIG. 4A is an expression detection result of necroptosis molecule in mouse intestinal epithelium under electron microscope scanning.

FIG. 4B shows results of western blot of necroptosis molecule in mouse intestinal epithelium.

FIG. 4C illustrates changes of protein levels of RIP3, MLKL and pMLKL, the key molecules of necroptoisis in intestinal epithelium of mouse with constitutive Prmt5 gene knockout displayed by immunohistochemistry.

FIG. 4D is a change result of protein levels of RIP3 and pMLKL, the key genes of necroptoisis in intestinal epithelial of mouse with Prmt5 gene knockout in CreERT system induced by tamoxifen displayed by immunohistochemistry.

FIG. 5 is a mouse lifetime analysis.

FIG. 6 is a histological analysis of mouse intestine.

FIG. 7 is a chromatogram of the polypeptide TAT-mRIP3.

FIG. 8 is a chromatogram of the polypeptide TAT-mRIP3-SDMA.

### DETAILED DESCRIPTION OF THE EMBODIMENTS

In order to make people in the technical field better understand the scheme of the disclosure, the technical scheme in the embodiment of the disclosure will be described clearly and completely with the attached drawings. Obviously, the described embodiment is only a part of the embodiment of the disclosure, but not all embodiments. Based on the embodiments in the present disclosure, all other embodiments obtained by ordinary technicians in the field without creative work should belong to the scope of protection of the present disclosure.

It should be noted that the terms “first” and “second” in the description and claims of the present disclosure and the above drawings are used to distinguish similar objects, and are not necessarily used to describe a specific order or sequence. It should be understood that the data thus used are interchangeable under appropriate circumstances, so that the embodiments of the disclosure described herein may be implemented in other orders than those illustrated or described herein. Furthermore, the terms “including” and “having” and any variations thereof are intended to cover non-exclusive inclusion, for example, a process, method, system, product or equipment that includes a series of steps or units is not necessarily limited to those explicitly listed, but may include other steps or units not explicitly listed or inherent to such processes, methods, products or equipment.

The present disclosure will be described in further detail with reference to the accompanying drawings.

### 1. Construction of Polypeptide Fragments

In order to verify the biological effect caused by mutation of amino acid at the 479th position of RIP3, GenScript Biotech Corporation is entrusted to synthesize the peptide fragments with symmetric di-methylation modification (TAT-MRIP3-SDMA) and unmodified (TAT-mRIP3) of arginine at 479th position in RIP 3 of mouse, as shown in Table 1 below.

TABLE 1

| | | |
|---|---|---|
| TAT | GRKKRRQRRRPQ | SEQ ID |
| | | NO: 3 |
| TAT-mRIP3 | GRKKRRQRRRPQQAQFGRGRGWQPFHK | SEQ ID |
| | | NO: 2 |
| TAT-mRIP3- | GRKKRRQRRRPQQAQFGRG (SDMA) | SEQ ID |
| SDMA | GWQPFHK | NO: 1 |

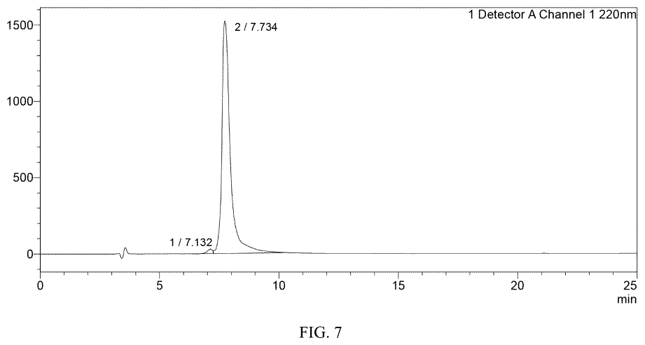

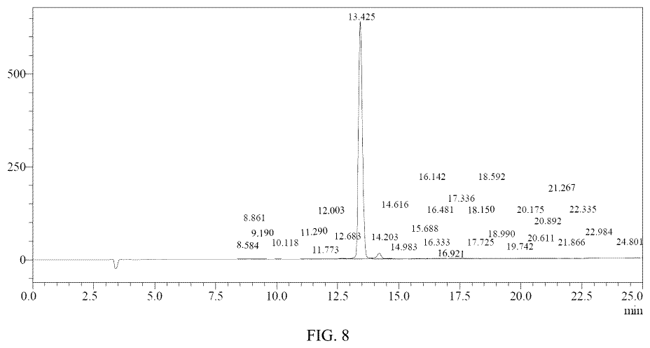

Among them, the chromatograms of polypeptide TAT-mRIP3 and polypeptide TAT-mRIP3-SDMA are shown in FIG. 7 and FIG. 8.

### 2. Membrane Penetrating Experiment

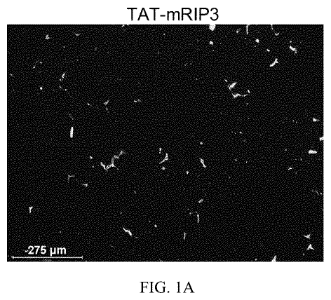

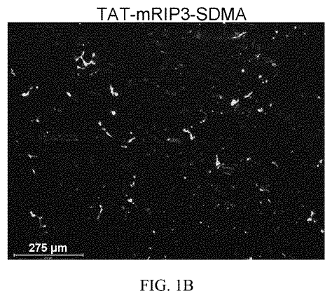

Under the condition of necroptosis activation, it is observed whether the symmetrical dimethyl modified polypeptide fragment is capable of reducing the necroptosis and protecting cells from death. 20 μM polypeptide is added to mouse L929 cells for 24 hours, and the efficiency of polypeptide entering the target cells is analyzed by fluorescence microscope, with the scale of 275 μm, and the results are shown in FIG. 1A-FIG. 1B. The results show the fluorescence intensity after the polypeptide is added for 24 hours, indicating that the efficiency of polypeptide fragments entering the cells through the cell membrane of the two groups is the same.

### 3. Necroptosis Stimulation Experiment

The necroptosis induced by TNF-α (20 ng/mL)+Z-VAD (20 μM) is in mouse fibroblast L929, and polypeptide (20 μM) is added to intervene. After 12 hours treatment, the morphological changes of the cell are analyzed: Dimethyl sulfoxide (DMSO) is used as control group); the scale is 20 μm.

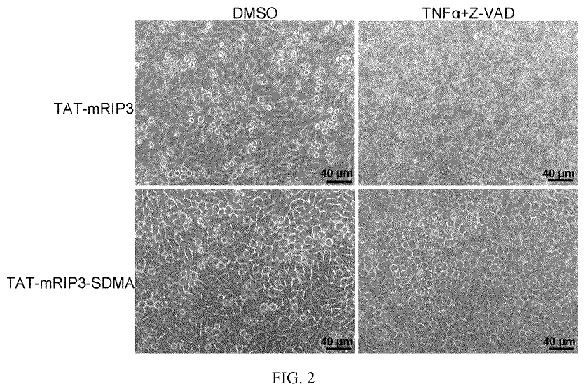

After 12 hours, the necroptosis of the cells is stimulated. After 5 hours, compared with the cells without drug stimulation, the cell morphology shows obvious death characteristics, that is, the cell morphology is changed from spindle to round. Compared with the polypeptide of TAT-mRIP3, the modified polypeptide TAT-mRIP3-SDMA has obvious protective effect, and the cell morphology is more complete (the result is shown in FIG. 2).

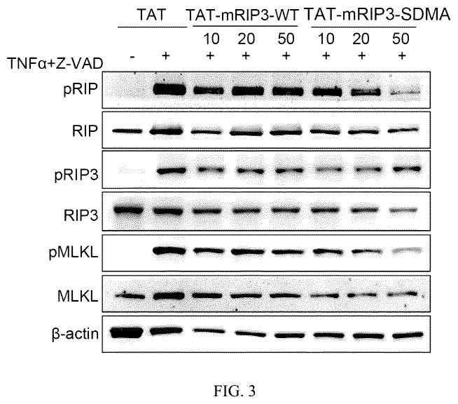

Furthermore, the necroptosis reaction in cells is detected. Mouse fibroblast L929 is treated with the classic cell necroptosis inducer TNF-α (20 ng/ml)+Z-VAD (20 μM), and at the same time, the peptides TAT (20 μM), TAT-mRIP3-WT and TAT-mRIP3-SDMA (10, 20 and 50 μM) are added. Cell samples are collected for protein detection after 12 hours. It is found that the polypeptide in TAT-mRIP3-SDMA group show protective effects at the concentrations of 10 μM, 20 μM and 50 μM, that is, the expression levels of pRIP1, pRIP3 and pMLKL are lower than control group (the results are shown in FIG. 3).

4. Expression Analysis of Necroptosis Signal Molecules in Mouse Intestinal Epithelial Tissue with Prmt5 Knockout

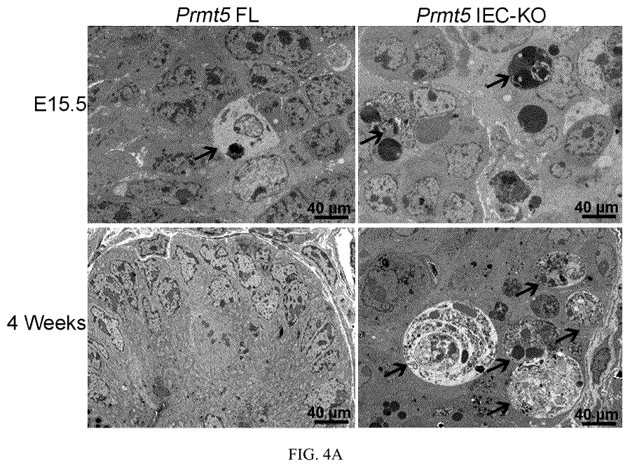

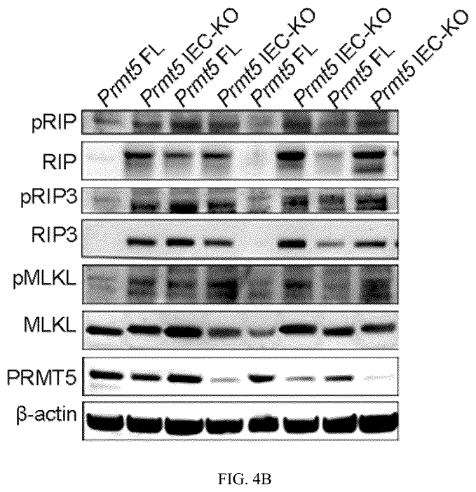

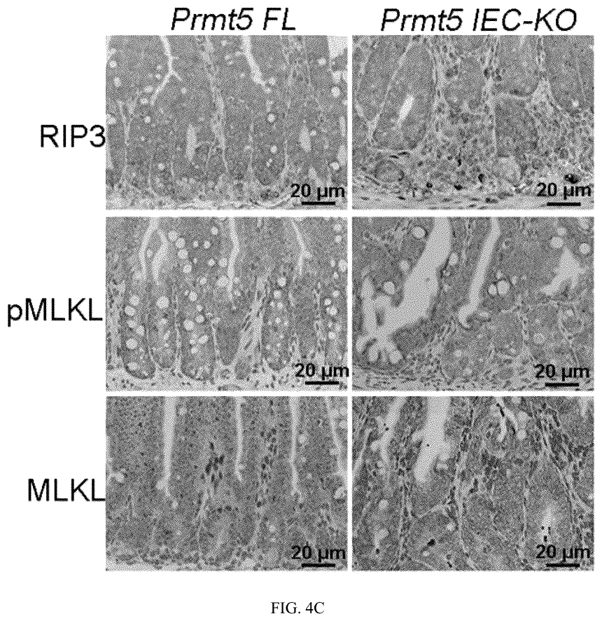

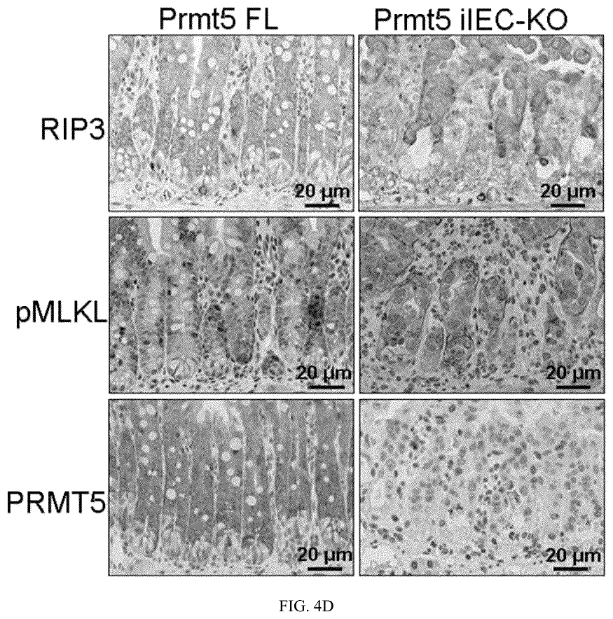

In order to clarify the microscopic changes in the intestinal epithelium of mice after Prmt5 knockout, the intestinal epithelium of mice is examined by transmission electron microscope. The results show that a large number of necroptosis cells appear in the intestinal epithelium of Prmt 5^{IEC-KO} of mouse at 4 weeks after birth and 15 days of embryonic stage, but this phenomenon is not found in the control group (FIG. 4A shows the ultrastructure of intestinal epithelium of mice at 15 days of embryonic stage and 4 weeks after birth). Based on the results of transmission electron microscope, it may be concluded that the activation of necroptosis in intestinal tissue may be the key link of intestinal abnormality after Prmt5 knockout.

To further verify the activation of necroptosis in the intestinal epithelium of mice, the intestinal epithelial tissue is collected, and the key molecules of necroptosis signal RIP1, pRIP1, RIP3, pRIP3, MLKL and pMLKL are detected. The results of western blot show that the key genes of necroptosis signal are obviously increased (as shown in FIG. 4B, protein level changes of the key molecules of necroptosis pathway RIP1, pRIP1, RIP3, pRIP3, MLKL and pMLKL are displayed.) The expression of RIP3, pMLKL and MLKL in Prmt5^{IEC-KO} mice is increased, which detected by protein immunostaining (as shown in FIG. 4C). Eight-week-old conditionally induced Prmt5^{iIEC-KO} mice (75 mg/kg) is treated with tamoxifen (intraperitoneal injection, once a day, for 5 days), and necroptosis key genes in the mouse have the same results after 9 days of injection (as shown in FIG. 4D, which verifies the knockout effect of Prmt5 in mice. The arrow in the figure indicates the necroptosis cells. The scale in the figure represents 20 μm). It can be seen that the disclosure proves that necroptosis-related molecules are activated and expressed in the intestinal epithelial tissue of mice with Prmt5 knockout from different perspectives.

### 5. Protective Effect of Di-Methylated Peptide Fragments

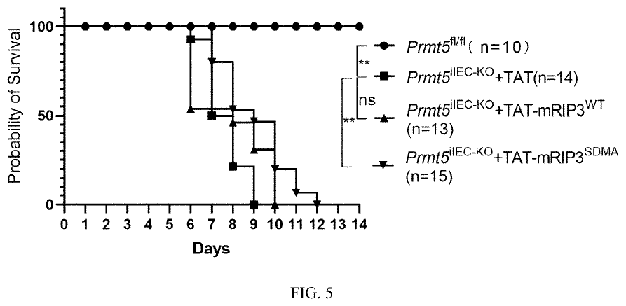

Tamoxifen-induced mouse intestinal epithelial cell-specific gene knockout mice (Prmt5^{iIEC-KO}) are used. Tamoxifen is given (75 mg/kg) to mice at age of 8 weeks to induce Prmt5 gene knockout and cell necroptosis (intraperitoneal injection, once a day, for 5 days). Using Prmt5^{iIEC-KO}, while tamoxifen is injected to induce Prmt5 gene knockout, mice are injected with different types and concentrations of peptides intraperitoneally, and the survival cycle of mice is analyzed, ** p<0.01. Mice are injected intraperitoneally with 10 mg/kg polypeptide once a day until the mice die. It is found that the lifetime of mice treated with TAT-mRIP3-SDMA polypeptide is significantly prolonged, suggesting a significant protective effect (the result is shown in FIG. 5).

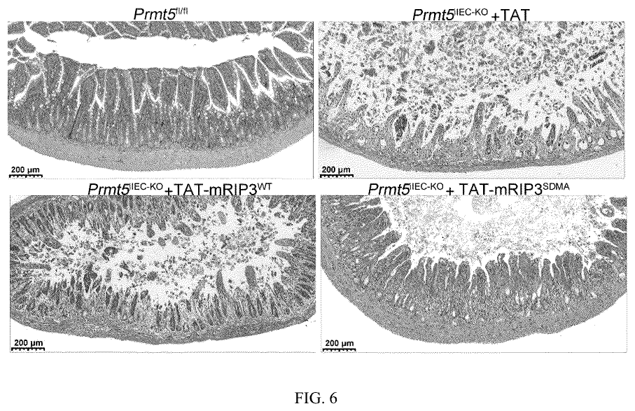

Furthermore, the intestinal tissues of mice are stained with hematoxylin-eosin (HE), and the results show that compared with TAT and unmodified (TAT-mRIP3) polypeptide group, TAT-mRIP3-SDMA polypeptide group has significant protective effect, which shows the intestinal tissue necrosis of mice is reduced and the intestinal cavity structure is basically intact (as shown in FIG. 6).

The above contents are only for explaining the technical idea of the present disclosure, and may not be used to limit the protection scope of the present disclosure. Any changes made on the basis of the technical scheme according to the technical idea proposed by the present disclosure fall within the protection scope of the claims of the present disclosure.

What is claimed is:

1. A polypeptide based on Receptor-Interacting Protein Kinase-3 (RIP3) methylation modification, formed by connecting a cell-penetrating peptide TAT and a methylated RIP3 polypeptide.

2. The polypeptide based on RIP3 methylation modification according to claim 1, wherein a 479th arginine of RIP3 protein is subjected to symmetric di-methylation modification.

3. The polypeptide based on RIP3 methylation modification according to claim 1, wherein an amino acid sequence of the polypeptide based on RIP3 methylation modification is shown in SEQ ID NO: 1.

4. The polypeptide based on RIP3 methylation modification according to claim 1, wherein a molecular weight of the polypeptide based on RIP3 methylation modification is 3821.27 Da.

5. An application of the polypeptide based on RIP3 methylation modification according to claim 1 in preparing a medicine for preventing and treating diseases caused by cell necroptosis.

6. The application according to claim 5, wherein the medicine is a medicine for increasing a resistance of cells to necroptosis.

7. The application according to claim 6, wherein the medicine is a medicine for reducing cell death and necroptosis.

8. The application according to claim 6, wherein the medicine is a medicine for reducing expression levels of necroptosis molecules pRIP1, pRIP3 and pMLKL.

9. An application of the polypeptide based on RIP3 methylation modification according to claim 1 in preparing a medicine for preventing and treating intestinal inflammatory injury.

10. An application of the polypeptide based on RIP3 methylation modification according to claim 1 in preparing a medicine for preventing or treating intestinal inflammatory injury.

11. A medicine for inhibiting intestinal inflammatory injury, prepared by the polypeptide based on RIP3 methylation modification according to claim 1 with pharmaceutically acceptable adjuvants.

12. A medicine for inhibiting intestinal inflammatory injury, prepared by the polypeptide based on RIP3 methylation modification according to claim 1 without pharmaceutically acceptable adjuvants.
